(19) United States
(12) Patent Application Publication
(10) Pub. No.: US 2007/0155961 A1
(43) Pub. Date: Jul. 5, 2007

(54) Method for preparing phthalocyanines

(75) Inventors: Thomas Gessner, Heidelberg (DE); Sophia Ebert, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Lidwigshafen (DE)

(21) Appl. No.: 10/584,631

(22) PCT Filed: Dec. 30, 2004
(86) PCT No.: PCT/EP04/14825

§ 371 Date: Jun. 26, 2006

(30) Foreign Application Priority Data
Jan. 8, 2004 (DE) 10 2004 001 457.4

(51) Int. Cl. C07D 487/22

(52) U.S. Cl. 540/124; 540/143; 540/125

(57) ABSTRACT

The present invention relates to a process for the preparation of metal-free phthalocyanines of the formula I 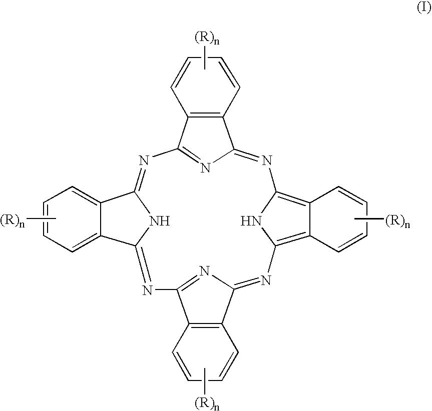
by conversion of an ortho-phthalodinitrile of the formula Ia 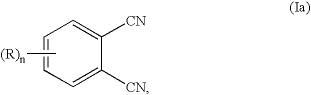
in an inert solvent with a boiling point of at least 120° C. (at standard pressure) in the presence of ammonia, in which, in formula I or Ia, the variable n can adopt values of 1, 2, 3 or 4 and the R radicals denote a five- or six-membered saturated nitrogen-comprising heterocyclic ring optionally substituted by one or two C₁-C₈-alkyl groups which is bonded via a ring nitrogen atom to the benzene ring and which can still comprise one or two additional nitrogen atoms or an additional oxygen or sulfur atom, which comprises carrying out the conversion in the presence of an alkali metal hydroxide or alkali metal carbonate.

The present invention relates to a process for the preparation of metal-free phthalocyanines of the formula I 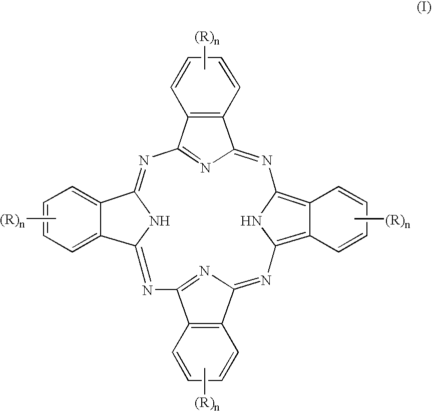
by conversion of an ortho-phthalodinitrile of the formula Ia 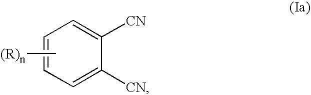
in an inert solvent with a boiling point of at least 120° C. (at standard pressure) in the presence of ammonia,
in which, in formula I or Ia, the variable n can adopt values of 1, 2, 3 or 4 and the R radicals denote a five- or six-membered saturated nitrogen-comprising heterocyclic ring optionally substituted by one or two C₁-C₈-alkyl groups which is bonded via a ring nitrogen atom to the benzene ring and which can still comprise one or two additional nitrogen atoms or an additional oxygen or sulfur atom,
which comprises carrying out the conversion in the presence of an alkali metal hydroxide or alkali metal carbonate.

The preparation of metal-free phthalocyanines is generally carried out in a high-boiling solvent starting from isoindoleninediimines, as, for example, disclosed in the document U.S. Pat. No. 3,509,146, or starting from o-phthalodinitrile or isoindoleninediimines in the presence of a base, for example ammonia, as, for example, explained in P. J. Brach, S. J. Grammatica, O. A. Ossanna and L. Weinberger, J. Heterocyclic Chem., 7 (1970), 1403-1405.

The preparation of metal-free phthalocyanines of the formula I using the preparation processes as represented in the abovementioned documents results, however, in unsatisfactory yields. Thus, for example, 1(4),8(11),15(18), 22(25)-tetra-(3-methylpiperidino)phthalocyanine can be obtained starting from 3-(3-methylpiperidino)phthalodinitrile according to the instructions of P. J. Brach et al. indeed with high purity but only in a low yield of 37%.

It was therefore an object of the present invention to make available a process by which metal-free phthalocyanines of the formula I can be prepared with high purity and in high yield. This object has been achieved by the process described at the start.

The R radicals of the formulae I and Ia are five- or six-membered saturated nitrogen-comprising heterocyclic rings optionally substituted by one or two C₁-C₈-alkyl groups which are bonded via a suitable ring nitrogen atom to the benzene ring and can still comprise one or two additional nitrogen atoms or an additional oxygen or sulfur atom.

The R radicals are preferably six-membered saturated nitrogen-comprising heterocyclic rings optionally substituted by one or two C₁-C₄-alkyl groups which are bonded via a ring nitrogen atom to the benzene ring and can still comprise an additional nitrogen atom.

Examples of such heterocyclic rings are pyrrolidin-1-yl, 2- or 3-methylpyrrolidin-1-yl, 2,4-dimethyl-3-ethylpyrrolidin-1-yl, pyrazolidin-1-yl, 2-, 3-, 4- or 5-methylpyrazolidin-1-yl, imidazolidin-1-yl, 2-, 3-, 4- or 5-methylimidazolidin-1-yl, oxazolidin-3-yl, 2-, 4- or 5-methyloxazolidin-3-yl, isoxazolidin-2-yl, 3-, 4- or 5-methylisoxazolidin-2-yl, piperidin-1-yl, (C₁-C₄-alkyl)piperidin-1-yl, such as 2-, 3-, 4-methyl- or -ethylpiperidin-1-yl, 2,6-dimethylpiperidin-1-yl, piperazin-1-yl, 4-(C₁-C₄-alkyl)piperazin-1-yl, such as 4-methyl- or 4-ethylpiperazin-1-yl, morpholin-4-yl, thiomorpholin-4-yl or S,S-dioxidothiomorpholin-4-yl.

R is particularly preferably piperidin-1-yl or piperazin-1-yl radicals substituted by one or two C₁-C₄-alkyl groups.

Examples of C₁-C₈ or C₁-C₄-alkyl groups as possible substituents of heterocyclic rings are methyl, ethyl, propyl, isopropyl, butyl, isobutyl, sec-butyl, tert-butyl, pentyl, isopentyl, neopentyl, tert-pentyl, hexyl, 2-methylpentyl, heptyl, hept-3-yl, octyl, 2-ethylhexyl and isooctyl.

The use of mixtures of different compounds of the formula Ia, which in each case differ from one another in the values of n and/or the chemical nature of their R radicals and/or the relative positions thereof with respect to the nitrile groups of the phthalodinitrile, is possible in principle according to the process.

However, the compound of formula la is preferably a pure compound with a given value of the variable n, the R radicals preferably being identical for n equal to 2, 3 or 4.

Particularly preferably, in formula I or Ia, the variable n adopts the value 1.

Mention may also be made in this connection that, not only for chemically different R radicals but also in the last preferred case of identical R radicals, the resulting compound of the formula I can consist of a mixture of positional isomers. This is explained by way of example in the following examples (cf. “B) Conversion in n-butyl glycol”).

All solvents known to a person skilled in the art from the state of the art for the preparation of metal-free phthalocyanines are possible as inert solvents provided that they have a boiling point of at least 120° C. (at standard pressure).

Use is preferably made, in the process according to the invention, of solvents chosen from the group consisting of ethylene glycol, diethylene glycol, propylene glycol, 1,2-butanediol, 1,3-butanediol, 1,4-butanediol, 2,3-butanediol, the mono- and di(C₁-C₄-alkyl) ethers of the abovementioned diols, 2-[di(C₁-C₄-alkyl)amino]ethanol and 3-[di(C₁-C₄-alkyl)amino]propanol. Suitable C₁-C₄-alkyl radicals of the mono- and di(C₁-C₄-alkyl) ethers of the abovementioned diols and of the 2-[di(C₁-C₄-alkyl)amino]-ethanols and 3-[di(C₁-C₄-alkyl)amino]propanols are methyl, ethyl, n-propyl, isopropyl, n-butyl, isobutyl, sec-butyl or tert-butyl. The alkyl radicals are, in the case of the mono- and diethers, generally methyl or n-butyl and, in the case of the aminoalcohols, methyl. Use is advantageously made of the monoethers of the abovementioned diols and 3-[di(C₁-C₄-alkyl)amino]propanols.

When choosing a specific solvent from the abovementioned group, the stipulation, that it must have a boiling point of at least 120° C., is naturally to be observed, in addition.

Particular preference is given to n-butyl glycol and 3-dimethylaminopropanol.

Use is preferably made, as alkali metal hydroxide or alkali metal carbonate, of sodium hydroxide, potassium hydroxide, sodium carbonate and potassium carbonate, particularly preferably of sodium hydroxide and potassium carbonate.

One of the abovementioned bases is normally added according to the process; however, mixtures of two or more bases can also be used.

The proportion of the base or base mixture is normally 0.5 to 10 mol %, preferably 1 to 6 mol %, based on the number of moles of the compound of formula Ia.

The conversion according to the invention is usually carried out in standard reactors with corresponding stirring devices and optionally internal fittings which improve the intermixing, such as, for example, baffles.

The ammonia is usually introduced into the reaction mixture at the bottom of the reactor at a constant volumetric flow rate. The amount of ammonia metered in per unit of time can in this connection be calibrated using conventional methods, e.g. by collecting in dilute acetic acid and subsequent titration.

The amount of ammonia is preferably at least two molar equivalents, based on the number of moles of compound of the formula Ia, since it is assumed that the ammonia acts catalytically in accordance with the following chemical equation:

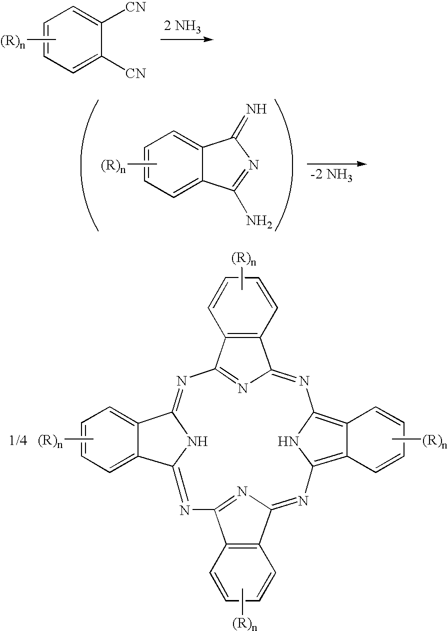

The duration of the introduction of the ammonia is usually several hours. In this connection, the test carried out by the Applicant Company on the laboratory scale may serve as information aid. For example, both in a 500 ml round-bottomed flask with a paddle stirrer and in a 2 l reactor with a disk mixer and baffles, the minimum amount of two molar equivalents of ammonia was achieved after a total duration of introduction of 9 hours (2 hours during the heat-up phase and 7 hours at the final temperature), the introduction of gas having been carried out at the bottom of the flask or reactor using a dip pipe.

The reaction temperature is usually between 140 and 170° C., yet the most suitable reaction temperature for a specific inert solvent can be determined by a person skilled in the art in a simple way by routine preliminary experiments. For example, in experiments of the Applicant Company with the solvents 3-dimethylaminopropanol and n-butyl glycol, the highest yields were determined at reaction temperatures of approximately 150° C. and 160° C. respectively.

The ratio of compound of the formula Ia (number of moles) to inert solvent (volumes) is usually approximately two moles to one liter; however, in the individual case, it is possible both to rise above and to fall below this value.

### EXAMPLES

### Preparation of 1(4),8(11),15(18),22(25)-tetra(3-methylpiperidino)phthalocyanine

A) Conversion in 3-dimethylaminopropanol:

225.3 g (1.00 mol) of 3-(3-methylpiperidino)phthalodinitrile were introduced into 500 ml of 3-dimethylaminopropanol in a 2 l flange flask at ambient temperature with stirring (150 revolutions/min). 4.85 g (0.035 mol; 3.5 mol %) of potassium carbonate were subsequently added. A total of 34.1 g (2.00 mol) of ammonia were introduced in the gaseous form into the reaction mixture over 9 hours (2 h during the heat-up phase and 7 h during the reaction phase) via a dip pipe with a volumetric flow rate of approximately 83 ml/min, the reaction mixture being heated to a final temperature of 150° C. and being maintained at this temperature for 15 hours. The black reaction solution was afterwards cooled to 50° C. and 1000 ml of methanol were added thereto within 2 hours with stirring, in order to completely precipitate the solid produced on cooling. The suspension was stirred for a further hour at 50° C., then cooled to ambient temperature and filtered on a suction filter. The filter cake was washed first with 800 ml of methanol and then with 1000 ml of water and finally pulled dry.

After drying under vacuum at 60° C., a black powder was obtained in a yield of 170.4 g (70% of theory, based on the pure substance).

A sample recrystallized three times from 3-dimethylaminopropanol and once from n-butyl glycol was virtually pure analytically. The elemental analysis produced:

| | | | | |
|---|---|---|---|---|
| C₅₆H₆₂N₁₂ | calc. | C 74.47% | H 6.92% | N 18.61% |
| (903.2 g/mol) | found | C 74.8% | H 6.9% | N 18.2% |

B) Conversion in n-butyl glycol:

n-Butyl glycol was used instead of 3-dimethylaminopropanol as inert solvent for the conversion and 160° C. was used as final temperature (with otherwise unchanged remaining parameters in comparison with the experimental procedure according to A)) and gave comparable yields and purities of the desired product, which (as also according to A) was present as a mixture of different positional isomers with the following structures:

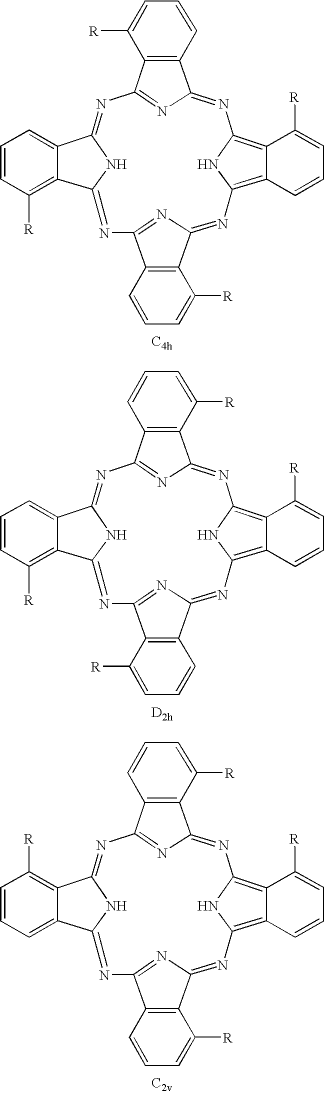

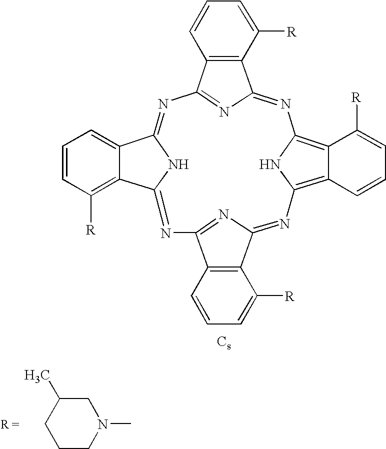

1. A process for the preparation of metal-free phthalocyanines of the formula I 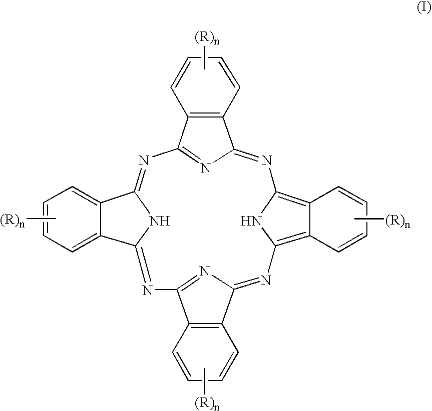
by conversion of an ortho-phthalodinitrile of the formula Ia 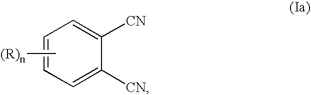
in an inert solvent with a boiling point of at least 120° C. (at standard pressure) in the presence of ammonia,
in which, in formula I or Ia, the variable n can adopt values of 1, 2, 3 or 4 and the R radicals denote a five- or six-membered saturated nitrogen-comprising heterocyclic ring optionally substituted by one or two C₁-C₈-alkyl groups which is bonded via a ring nitrogen atom to the benzene ring and which can still comprise one or two additional nitrogen atoms or an additional oxygen or sulfur atom,
which comprises carrying out the conversion in the presence of an alkali metal hydroxide or alkali metal carbonate.

2. The process according to claim 1, wherein the inert solvent is chosen from the group consisting of ethylene glycol, diethylene glycol, propylene glycol, 1,2-butanediol, 1,3-butanediol, 1,4-butanediol, 2,3-butanediol, the mono- and di(C₁-C₄-alkyl) ethers of the abovementioned diols, 2-[di(C₁-C₄-alkyl)amino]ethanol and 3-[di(C₁-C₄-alkyl)amino]propanol.

3. The process according to claim 1, wherein 3-dimethylaminopropanol or n-butyl glycol is used as inert solvent.

4. The process according to claim 1, wherein sodium hydroxide, potassium hydroxide, sodium carbonate or potassium carbonate are used as alkali metal hydroxide or alkali metal carbonate.

5. The process according to claim 1, wherein n in the formulae I and Ia adopts the value 1.

6. The process according to claim 1, wherein the R radicals denote a six-membered saturated nitrogen-comprising heterocyclic ring substituted by one or two C₁-C₄-alkyl groups which is bonded via a ring nitrogen atom to the benzene ring and which can still comprise an additional nitrogen atom.

7. The process according to claim 7, wherein the R radicals denote a piperidine or piperazine ring substituted by one or two C₁-C₄-alkyl groups which is bonded via the ring nitrogen atom or one of the two ring nitrogen atoms to the benzene ring.
